(19) United States
(12) Patent Application Publication
(10) Pub. No.: US 2026/0108698 A1
(43) Pub. Date: Apr. 23, 2026

(54) Aromatherapy candle lamp

(71) Applicant: Dongsheng Liang, Heyuan (CN)

(72) Inventor: Dongsheng Liang, Heyuan (CN)

(21) Appl. No.: 18/919,293

(22) Filed: Oct. 17, 2024

(51) Int. Cl. A61M 21/02; A61M 21/00

(52) U.S. Cl. CPC A61M 21/02; A61M 2021/0016; A61M 2205/3592; A61M 2205/587; A61M 2205/59; A61M 2205/8206

(57) ABSTRACT

An aromatherapy candle lamp is disclosed. The aromatherapy candle lamp relates to the technology of candle lamp, in an embodiment includes a shell, a base, an aromatherapy mechanism, a lighting mechanism and a battery box. A base is provided at the bottom of the shell, a battery box is provided in the base, the aromatherapy mechanism is detachably arranged on the battery box, a round hole connected to the inside of the shell is provided at the upper part of the shell, the upper end of the aromatherapy mechanism is located in the round hole, a lighting mechanism is provided on the aromatherapy mechanism, a power switch is provided on the base, the power switch, the lighting mechanism and the battery box are connected through a line.

### FIELD OF THE INVENTION

The present application relates to candle lamp. More specifically, the present application relates to an aromatherapy candle lamp.

### BACKGROUND

As the quality of life improves, people have higher expectations for the comfort and aesthetics of their homes. Aromatherapy products have gained popularity for their ability to enhance air quality and create a relaxed atmosphere. Additionally, candle lamps are commonly used as a lighting option, and there have been various designs developed in lighting equipment technology.

The combination of aromatherapy and candle lamps can create a comfortable atmosphere through the interaction of aromatherapy fragrance and light, making it very popular. The existing design of aromatherapy candle lamps involves adding a container in the middle of the candle, placing a sponge stick in the container, and then adding aromatherapy essential oils or solids to wet the sponge stick. This allows the aromatherapy essential oils to emit fragrance as air flows through. Additionally, there are candle lamps with controller timing for easy control, enabling more accurate regulation of fragrance release and light interaction.

The current aromatherapy candle lamps have some issues during use. Firstly, traditional aromatherapy candle lamps may produce a large amount of smoke, which not only affects air quality but also poses health risks. Secondly, while electric heating candle lamps can reduce smoke generation, their heating elements are easily corroded by aromatherapy essential oils, affecting their lifespan. Lastly, existing candle lamps have limitations in adjusting fragrance release and cannot meet diverse needs.

Therefore, a kind of aromatherapy candle lamp is designed in view of the above situation.

### SUMMARY

The technical problem to be solved by the present application is to overcome the defects of the above-mentioned technology and provide an aromatherapy candle lamp.

In an embodiment, to solve the above technical problems, the aromatherapy candle lamp, comprising a shell, a base, an aromatherapy mechanism, a lighting mechanism and a battery box, wherein the bottom of the shell is provided with a base, the battery box is provided in the base, the aromatherapy mechanism is detachably arranged on the battery box, the upper part of the shell is provided with a round hole connected to the inside of the shell, the upper end of the aromatherapy mechanism is located in the round hole, the aromatherapy mechanism is provided with a lighting mechanism, the base is provided with a power switch, and the power switch, the lighting mechanism and the battery box are connected by a line.

The aromatherapy mechanism comprises a container, a bottom cover and a cotton swab, wherein the bottom cover is detachably mounted on the bottom of the container, a cylinder is disposed at the upper end of the container, the cylinder is placed in a round hole, a cotton swab hole connected to the container is disposed at the top of the container, the cotton swab hole is located in the cylinder, the cotton swab is mounted in the cotton swab hole, and the lower end extends to the bottom of the container.

The lighting mechanism comprises a light source chip and a lamp, wherein the light source chip and the lamp are connected by a circuit.

A mounting groove is provided on one side of the container, the light source chip is arranged at the mounting groove, a mounting hole is provided on the top of the mounting groove, the mounting hole extends into the cylinder, and the lamp is arranged in the mounting hole.

A mounting base is provided in the cylinder at the upper end of the container, and a candle flame piece is inserted in the mounting base. The lamp and the candle flame piece cooperate, and the light shines on the candle flame piece and flickers left and right to simulate a real flame.

The remote control mechanism also includes a remote control mechanism, the remote control mechanism and the lighting mechanism cooperate.

As an improvement, a plug-in rod is provided at the bottom of the bottom cover, a card seat is provided at the upper end of the battery box, and the lower end of the plug-in rod is installed in the card seat.

As an improvement, the upper end of the bottom cover is provided with a circular groove, and the lower end of the container is arranged in the circular groove.

As an improvement, the remote control mechanism includes an infrared remote control and an infrared remote control receiver matched therewith, and the infrared remote control receiver is arranged on the light source chip.

As an improvement, a battery cover is provided at the lower end of the battery box.

The advantages of the present application compared with the prior art are:
1. To prevent smoke generation, the candle lamp in question has a cotton swab hole at the top connected to the container, where a cotton swab is placed. Aromatherapy essential oil or aromatherapy solid is then added to the container, and the fragrance is released through the cotton swab. This method effectively prevents smoke from being produced during the burning process, thus enhancing air quality and being more beneficial to human health.
2. To extend the service life, the candle lamp in this invention uses a static physical diffusion method to vaporize aromatherapy essential oil or aromatherapy solid, thus avoiding the issue of the heating element in traditional candle lamps being easily corroded by aromatherapy essential oil.
3. The bottom of the container has a detachable cover, making it easy to add your favorite essential oils or solid aromatherapy products. It is also easy to replace, providing more options for adjusting the release of the scent.
4. The candle lamp's remote control mechanism works with the lighting system and includes an infrared remote control function. This allows for precise control over the flickering of the lights and the timing of the lighting using the remote control, enhancing the realism of the flame.
5. The container of the candle lamp has an upper cylinder with a mounting base, and a candle flame piece is placed in the mounting base. The candle flame piece is easy to insert, install, and is cost-effective.

There is a mounting hole at the top of the mounting groove, which extends into the cylinder. The lamp is placed in the mounting hole, allowing the lamp beads to be inserted into the bottom of the blades. This creates a flashing light effect that simulates real flames, with the lights moving left and right. This physical mounting not only enhances the product's aesthetics but also increases its practicality.
6. Convenient cleaning and maintenance: The bottom cover of the candle lamp of the present invention can be disassembled, which is convenient for users to take out the aromatherapy mechanism and clean and maintain the internal container, thereby extending the service life of the product.

### BRIEF DESCRIPTION OF THE DRAWINGS

The technology may be more completely understood in connection with the following drawings, in which:

FIG. 1 is a front view of an aromatherapy candle lamp, according to an embodiment;

FIG. 2 is a perspective view of an aromatherapy candle lamp, according to an embodiment;

FIG. 3 is another perspective view of an aromatherapy candle lamp, according to an embodiment;

FIG. 4 is a perspective view of the shell of an aromatherapy candle lamp, according to an embodiment;

FIG. 5 is a perspective view of the internal structure of an aromatherapy candle lamp, according to an embodiment;

FIG. 6 is another perspective view of the internal structure of an aromatherapy candle lamp, according to an embodiment;

FIG. 7 is a sectional view, of portion A-A, of FIG. 1, thereof; and

FIG. 8 is an enlarged view, of circled portion B, of FIG. 7.

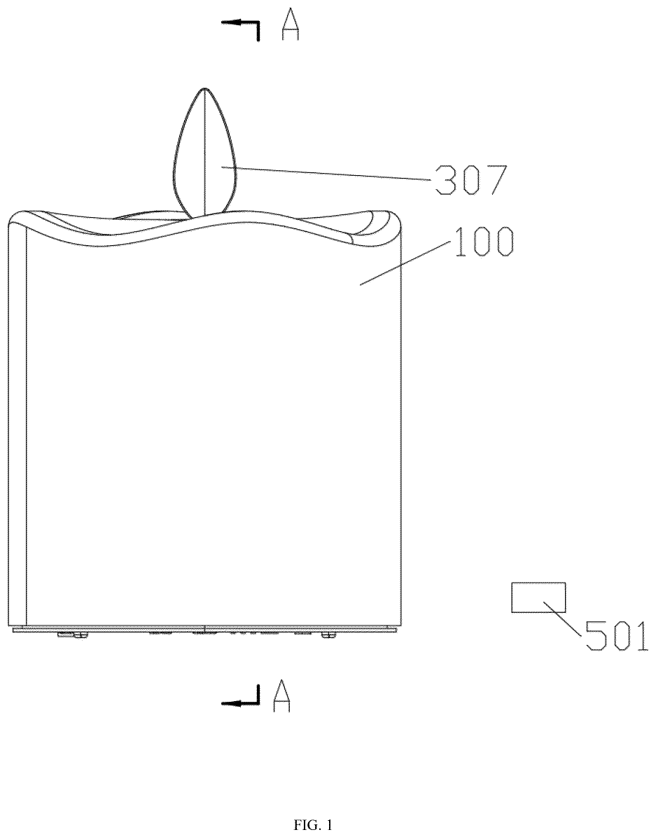

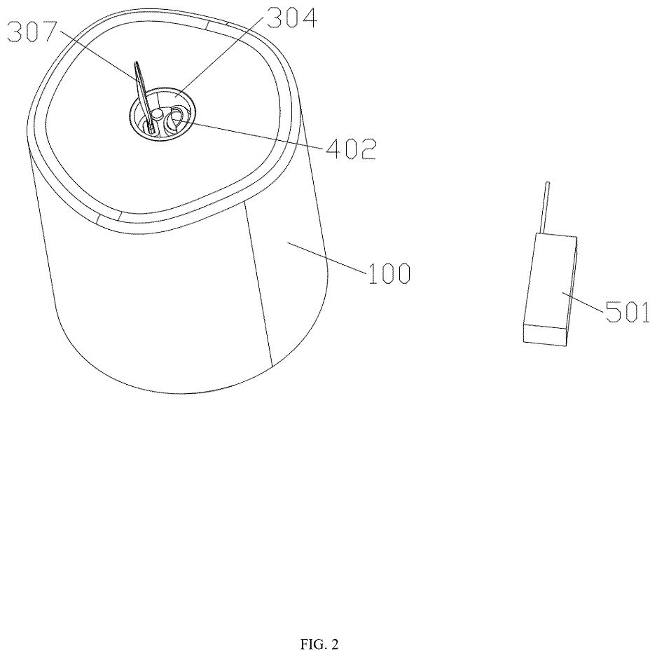

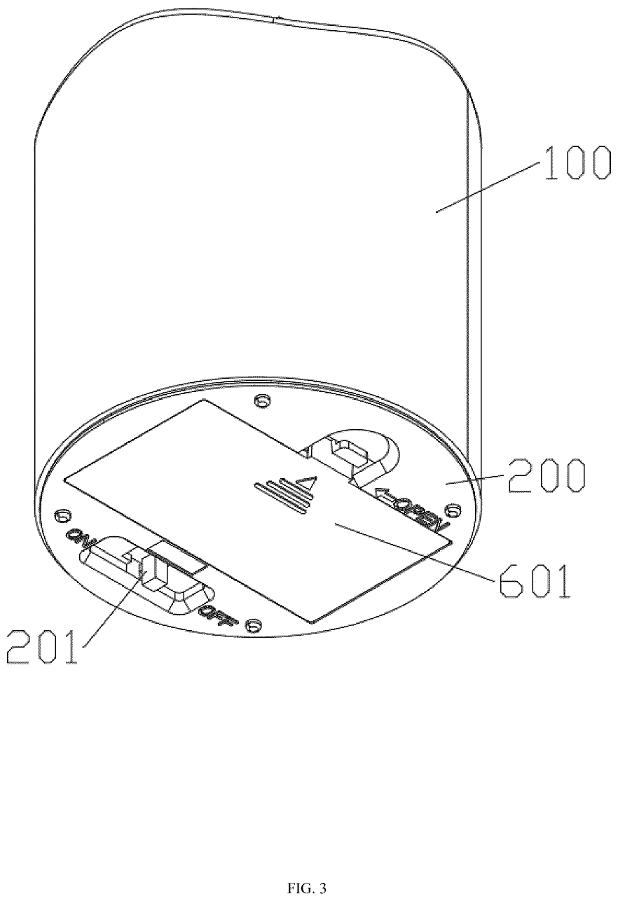

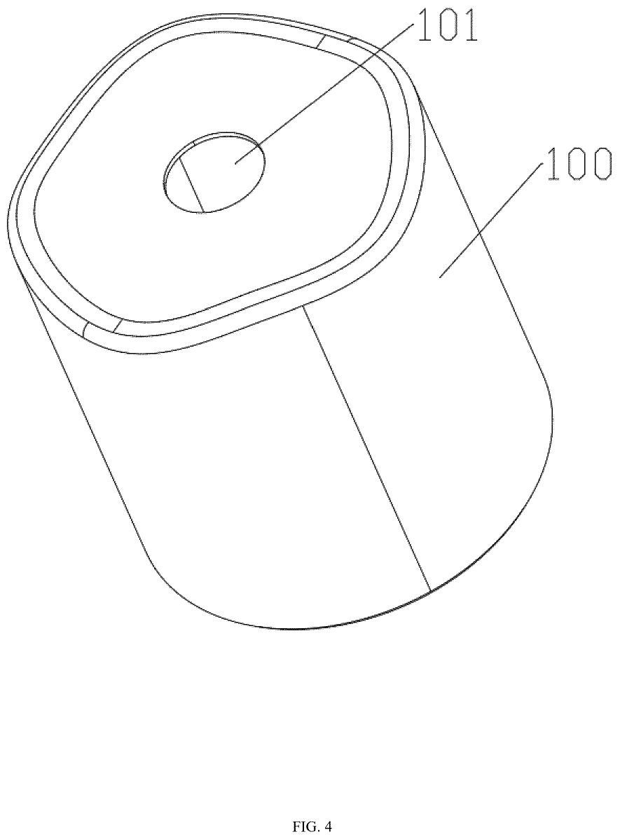

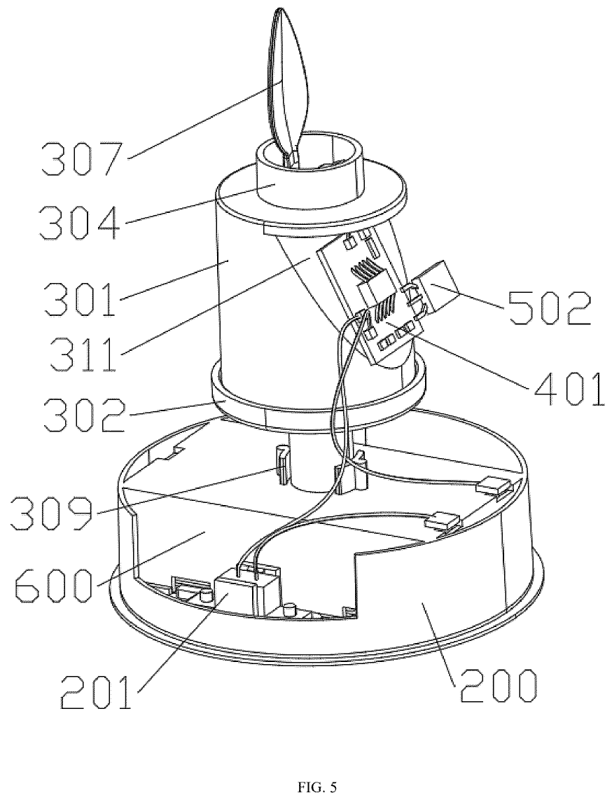

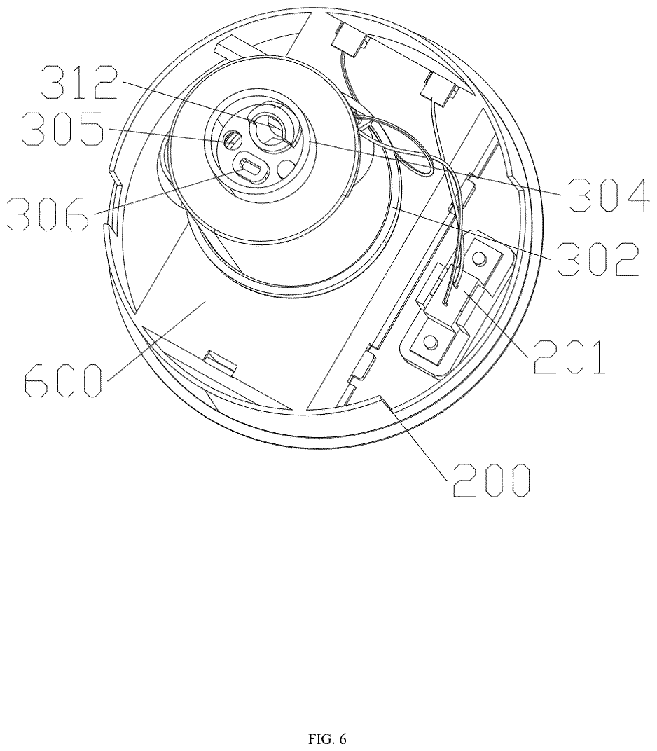

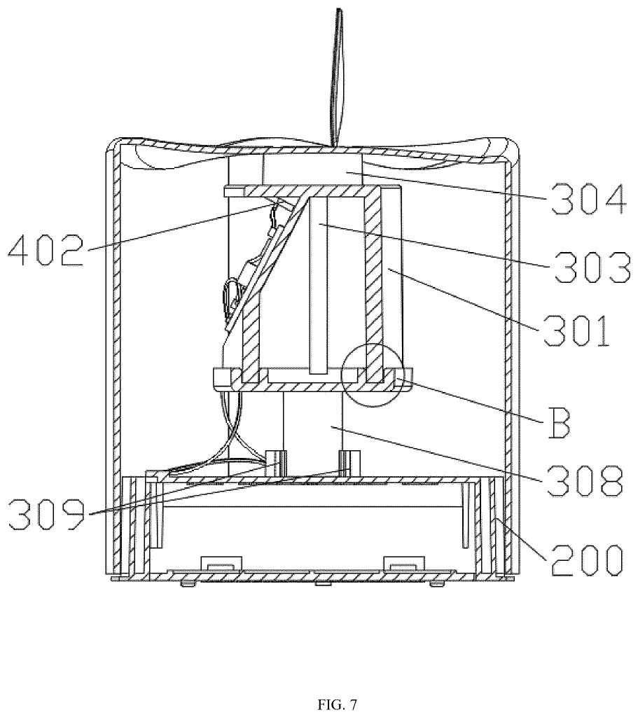

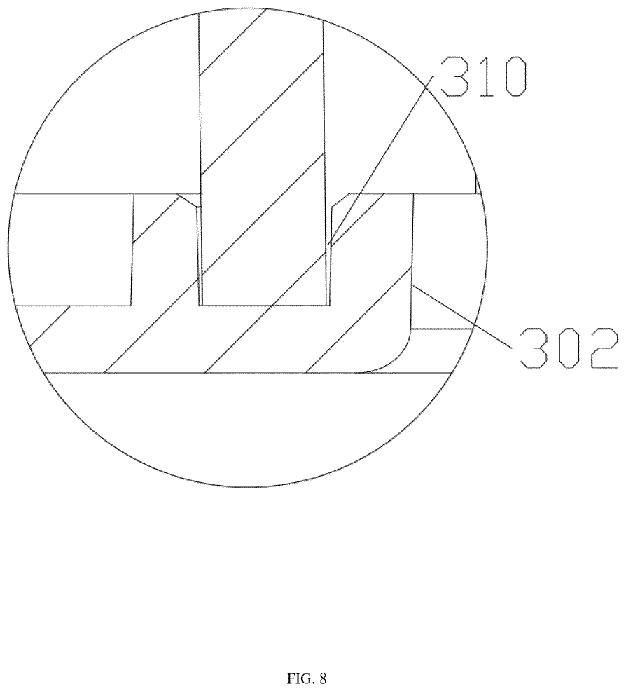

As shown in FIGS. 1-8
100. housing
101. round hole
200. base
201. power switch
301. container
302. bottom cover
303. cotton swab
304. cylinder
305. cotton swab hole
306. mounting base
307. candle flame sheet
308. plug-in rod
309. card base
310. circular groove
311. mounting groove
312. mounting hole
401. light source chip
402. light
501. infrared remote control
502. infrared remote-control receiver
600. battery box
601. battery cover

While the technology is susceptible to various modifications and alternative forms, specifics thereof have been shown by way of example and drawings, and will be described in detail. It should be understood, however, that the application is not limited to the particular embodiments described. On the contrary, the application is to cover modifications, equivalents, and alternatives falling within the spirit and scope of the technology.

### DETAILED DESCRIPTION OF EMBODIMENTS

The embodiments of the present technology described herein are not intended to be exhaustive or to limit the technology to the precise forms disclosed in the following detailed description. Rather, the embodiments are chosen and described so that others skilled in the art can appreciate and understand the principles and practices of the present technology.

All publications and patents mentioned herein are hereby incorporated by reference. The publications and patents disclosed herein are provided solely for their disclosure. Nothing herein is to be construed as an admission that the inventors are not entitled to antedate any publication and/or patent, including any publication and/or patent cited herein.

As shown in FIGS. 1-8, an aromatherapy candle lamp, comprising a shell 100, a base 200, a aromatherapy mechanism, a lighting mechanism and a battery box 600. The shell 100 is the outer covering body of the overall structure, the base 200 is provided at the bottom of the shell 100, and the base 200 can be removed from the shell 100 so as to clean and repair the internal structure; the battery box 600 is provided in the base 200, the aromatherapy mechanism is detachably arranged on the battery box 600, the upper part of the shell 100 is provided with a circular hole 101 connected to the inside of the shell 100, the upper end of the aromatherapy mechanism is located in the circular hole 101, the aromatherapy mechanism is provided with a lighting mechanism, the base 200 is provided with a power switch 201, the power switch 201, the lighting mechanism and the battery box 600 are connected by a line

The aromatherapy mechanism comprises a container 301, a bottom cover 302 and a cotton swab 303. The bottom cover 302 is detachably mounted on the bottom of the container 301. The container 301 and the bottom cover 302 together constitute a mechanism for containing aromatherapy essential oils or aromatherapy solids. A cylinder 304 is provided at the upper end of the container 301. The cylinder 304 is placed in the round hole 101. A cotton swab hole 305 connected to the container 301 is provided at the top of the container 301. The cotton swab hole 305 is located in the cylinder 304. The cotton swab 303 is installed in the cotton swab hole 305, and the lower end extends to the bottom of the container 301 to fully absorb the aromatherapy essential oils or aromatherapy solids in the container.

The lighting mechanism includes a light source chip 401 and a light 402, and the light source chip 401 and the light 402 are connected by a line;

A mounting groove 311 is provided on one side of the container 301, the light source chip 401 is arranged at the mounting groove 311, a mounting hole 312 is provided on the top of the mounting groove 311, the mounting hole 312 extends into the cylinder 304, and the light 402 is arranged in the mounting hole 312 to provide a light source;

A mounting base 306 is provided in the cylinder 304 at the upper end of the container 301, and a candle flame sheet 307 is inserted in the mounting base 306. The light 402 cooperates with the candle flame sheet 307, and the light 402 is located at the bottom of the candle flame sheet 307. The light can be shone on the candle flame sheet 307 and flickered left and right to simulate a real flame, which complements the aromatherapy function and creates a comfortable home environment together.

The remote-control mechanism also includes a remote-control mechanism, the remote-control mechanism and the lighting mechanism cooperate.

A plug-in rod 308 is provided at the bottom of the bottom cover 302, a card base 309 is provided at the upper end of the battery box 600, and the lower end of the plug-in rod 308 is installed in the card base 309, so as to realize the stable installation and quick disassembly of the aromatherapy mechanism.

The upper end of the bottom cover 302 is provided with a circular groove 310, and the lower end of the container 301 is arranged in the circular groove 310, which further enhances the stability of the connection and is also convenient for disassembly and installation. It is convenient to place the aromatherapy essential oil or aromatherapy solid of your favorite flavor into the container, and it is also convenient for replacement.

The remote-control mechanism includes an infrared remote control 501 and an infrared remote-control receiver 502 that cooperates therewith. The infrared remote-control receiver 502 is disposed on the light source chip 401, and the light timing and light 402 can be controlled by the infrared remote control 501.

The battery box 600 has a battery cover 601 at the lower end.

When in use, the user can unplug the base 200 from the shell 100, so that the internal mechanisms such as the aromatherapy mechanism and the lighting mechanism can be disassembled simultaneously. Then the plug-in rod 308 can be pulled out of the card seat 309, and the bottom cover 302 of the container 301 can be removed. Then the essential oil or aromatherapy solid can be put into the container 301. Then the bottom cover 302 can be covered and tightened, and then the aromatherapy mechanism and the lighting mechanism can be installed into the shell 100. The cylinder 304 can be placed in the round hole 101, so that the light 402, the top of the cotton swab 303, and the candle flame sheet 307 can all be exposed, and the base 200 can be installed to the bottom of the shell 100. After installation is completed.

Aromatherapy essential oil or aromatherapy solid will naturally evaporate through the cotton swab 303.

At the same time, by pressing the power switch 201, the light timing and light of the light 402 can be controlled by the infrared remote control 501. The light flickers left and right on the candle flame sheet 307 to simulate a real flame, which complements the aromatherapy function and adds a warm and romantic touch to the home environment.

1. An aromatherapy candle lamp, comprising a shell, a base, an aromatherapy mechanism, a lighting mechanism and a battery box, wherein the bottom of the shell is provided with a base, the battery box is provided in the base, the aromatherapy mechanism is detachably arranged on the battery box, the upper part of the shell is provided with a round hole connected to the inside of the shell, the upper end of the aromatherapy mechanism is located in the round hole, the aromatherapy mechanism is provided with a lighting mechanism, the base is provided with a power switch, and the power switch, the lighting mechanism and the battery box are connected by a line;
wherein the aromatherapy mechanism comprises a container, a bottom cover and a cotton swab, wherein the bottom cover is detachably mounted on the bottom of the container, a cylinder is disposed at the upper end of the container, the cylinder is placed in a round hole, a cotton swab hole connected to the container is disposed at the top of the container, the cotton swab hole is located in the cylinder, the cotton swab is mounted in the cotton swab hole, and the lower end extends to the bottom of the container;
the lighting mechanism comprises a light source chip and a lamp, wherein the light source chip and the lamp are connected by a circuit;
a mounting groove is provided on one side of the container, the light source chip is arranged at the mounting groove, a mounting hole is provided on the top of the mounting groove, the mounting hole extends into the cylinder, and the lamp is arranged in the mounting hole;
a mounting seat is provided in the cylinder at the upper end of the container, a candle flame piece is inserted in the mounting seat, and the lamp and the candle flame piece cooperate, and the light shines on the candle flame piece and flickers left and right to simulate a real flame;
the remote control mechanism also includes a remote control mechanism, the remote control mechanism and the lighting mechanism cooperate.

2. The aromatherapy candle lamp of claim 1, wherein the bottom of the bottom cover is provided with a plug-in rod, the upper end of the battery box is provided with a card seat, and the lower end of the plug-in rod is installed in the card seat.

3. The aromatherapy candle lamp of claim 2, wherein the upper end of the bottom cover is provided with a circular groove, and the lower end of the container is arranged in the circular groove.

4. The aromatherapy candle lamp of claim 1, wherein the remote control mechanism includes an infrared remote controller and an infrared remote control receiver matched therewith, and the infrared remote control receiver is arranged on the light source chip.

5. The aromatherapy candle lamp of claim 1, wherein the lower end of the battery box is provided with a battery cover.
